SEQUENCE LISTING (2 sequences)

SEQ ID NO: 1 - protein, 107 aa, Artificial Sequence
  hBU12 light chain variable region
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKR

SEQ ID NO: 2 - protein, 120 aa, Artificial Sequence
  hBU12 heavy chain variable region
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSS

(19) United States
(12) Patent Application Publication
(10) Pub. No.: US 2020/0230254 A1
(43) Pub. Date: Jul. 23, 2020

(54) COMBINATION THERAPY USING A CD19-ADC AND RCHP

(71) Applicant: SEATTLE GENETICS, INC., Bothell, WA (US)

(72) Inventor: Ana Kostic, Seattle, WA (US)

(73) Assignee: SEATTLE GENETICS, INC., Bothell, WA (US)

(21) Appl. No.: 16/319,147

(22) PCT Filed: Jul. 21, 2017
(86) PCT No.: PCT/US2017/043232

§ 371 Date: Jan. 18, 2019

Related U.S. Application Data
(60) Provisional application No. 62/365,796, filed on Jul. 22, 2016.

(51) Int. Cl. A61K 47/68; A61K 39/395; A61P 35/00; A61K 31/573; A61K 31/675; A61K 31/475; A61K 31/704

(52) U.S. Cl. CPC A61K 47/6803; A61K 47/6867; A61K 39/3955; A61P 35/00; A61K 2039/545; A61K 31/675; A61K 31/475; A61K 31/704; A61K 31/573

(57) ABSTRACT

This invention relates to treatment of Diffuse Large B-Cell Lymphoma (DLBCL) and Follicular Lymphoma (FL). Specifically, the disclosure provides a method of treating a subject having DLBCL or FL, the method comprising administering to the subject a drug combination consisting essentially of a CD19 antibody drug conjugate (CD19-ADC), and rituximab, cyclophosphamide, doxorubicin, and prednisone (RCHP).

### CROSS-REFERENCES TO RELATED APPLICATIONS

This application claims the benefit of U.S. Provisional App. No. 62/365,796 filed Jul. 22, 2016, which is hereby incorporated by reference in its entirety for all purposes.

### REFERENCE TO A SEQUENCE LISTING

This application includes an electronic sequence listing designated 0019-00611PC Sequence Listing ST25 created on Jul. 11, 2017 and containing 3 KB, which is hereby incorporated by reference.

### FIELD OF THE INVENTION

This invention relates to treatment of Diffuse Large B-Cell Lymphoma (DLBCL) and Follicular Lymphoma (FL).

### BACKGROUND OF THE INVENTION

CD19 is a member of the immunoglobulin superfamily. See, e.g., Tedder & Isaacs, J Immunol, 143: 712-717 (1989) and Del Nagro et al., Immunol Res, 31: 119-131 (2005). It is a B cell-specific marker not known to be expressed by any cell outside of the B lineage. CD19 expression is maintained upon malignant transformation, thus, CD19 is found on malignant cells in the majority of patients with B-cell leukemia or non-Hodgkin lymphoma. See, e.g., Nadler et al.,J Immunol, 131: 244-250 (1983); Anderson et al., Blood, 63: 1424-1433 (1984); and Scheuermann & Racila, Leuk Lymphoma, 18: 385-397 (1995).

Denintuzumab Mafodotin (SGN-CD19A) is a CD19-directed antibody-drug conjugate (ADC) consisting of three components: 1) the humanized antibody hBU12, specific for human CD19, 2) the microtubule disrupting agent, monomethyl auristatin F (MMAF), and 3) a stable linker, maleimidocaproyl, that covalently attaches MMAF to hBU12. The proposed mechanism of action (MOA) is initiated by SGN-CD19A binding to CD19 on the cell surface followed by internalization of the ADC. Upon trafficking to lysosomes, the delivered drug (cysmcMMAF) is released through proteolytic degradation of the antibody carrier. Binding of the released drug to tubulin disrupts the microtubule network, leading to cell cycle arrest and apoptosis.

SGN-CD19A activity has recently been assessed in a phase 1 clinical trial for treatment of patients with relapsed or refractory B-lineage non-Hodgkin lymphoma (B-NHL). However, improvements are needed in cancer therapy. The present invention solves this and other problems.

### BRIEF SUMMARY OF THE INVENTION

The present disclosure provides a method of treating a subject having DLBCL or FL, by administering a drug combination consisting essentially of a CD19 antibody drug conjugate (CD19-ADC) and RCHP (a combination of rituximab, cyclophosphamide, doxorubicin and prednisone). The present disclosure also provides a method of treating a subject having DLBCL or FL, by administering a drug combination consisting essentially of a CD19 antibody drug conjugate (CD19-ADC) and RCHOP (a combination of rituximab, cyclophosphamide, doxorubicin, vincristine and prednisone). In one embodiment, DLBCL or FL in the subject has not previously been treated. In another embodiment, DLBCL or FL in the subject has previously been treated.

In one aspect, the CD19-ADC is preferably SGN-CD19A, i.e., a humanized hBU12 antibody conjugated to a maleimidocaproyl monomethyl auristatin F (mcMMAF) molecule.

### DETAILED DESCRIPTION OF THE INVENTION

### Definitions

The term “CD19” refers to “cluster of differentiation protein 19”, a human protein that is expressed on human B cells. The amino acid sequence of human CD19 is known and is disclosed, e.g., at NCBI Reference Sequence: NP_001171569.1.

The term “RCHOP” refers to a combination of an anti-cancer antibody (rituximab) and a chemotherapeutic therapy consisting of cyclophosphamide, doxorubicin, vincristine and prednisone.

The term “RCHP” refers to a combination of an anti-cancer antibody (rituximab) and a chemotherapeutic therapy consisting of cyclophosphamide, doxorubicin and prednisone.

A “disorder”, as used herein, and the terms “CD19-associated disorder” and “CD19-associated disease” refer to any condition that would benefit from treatment with a CD19-antibody drug conjugate (CD19-ADC), such as SGN-CD19A, as described herein. This includes chronic and acute disorders or diseases including those pathological conditions that predispose the mammal to the disorder in question. Non-limiting examples or disorders to be treated herein include CD19 expressing cancers, including hematological malignancies, benign and malignant tumors, leukemias and lymphoid malignancies, as well as inflammatory, angiogenic and immunologic disorders. Specific examples of disorders are disclosed infra.

The terms “treatment” and “therapy”, and the like, as used herein, are meant to include therapeutic or suppressive measures for a disease or disorder leading to any clinically desirable or beneficial effect, including, but not limited to, alleviation or relief of one or more symptoms, regression, slowing or cessation of progression of the disease or disorder. For example, treatment can include a decrease or elimination of a clinical or diagnostic symptom of a CD19-expressing disorder after the onset of the clinical or diagnostic symptom by administration of an anti-CD19 antibody or other CD19 binding agent to a subject. Treatment can be evidenced as a decrease in the severity of a symptom, the number of symptoms, or frequency of relapse.

Except when noted, the terms “subject” or “patient” are used interchangeably and refer to mammals such as human patients and non-human primates, as well as experimental animals such as rabbits, dogs, cats, rats, mice, and other animals. Accordingly, the term “subject” or “patient” as used herein means any mammalian patient or subject to which the CD19 binding agents of the invention can be administered. In preferred embodiments, the terms subject or patient are used to refer to human patients. Subjects of the present invention include those that have been diagnosed with a CD19 expressing cancer, including, for example, B cell lymphoma or B cell leukemia, including, but not limited to, non-Hodgkin lymphoma, chronic lymphocytic leukemia, and acute lymphoblastic leukemia.

A subject with a refractory CD19 expressing cancer is a subject who does not respond to therapy, i.e., the subject continues to experience disease progression despite therapy.

A subject with a relapsed CD19 expressing cancer is a subject who has responded to the therapy at one point, but has had a recurrence or further progression of disease following the response.

The term “effective amount” refers to the amount of a CD19-ADC, e.g., SGN-CD19A, that is sufficient to inhibit the occurrence or ameliorate one or more clinical or diagnostic symptoms of a CD19-associated disorder in a subject. An effective amount of an agent is administered according to the methods described herein in an “effective regimen.” The term “effective regimen” refers to a combination of amount of the agent and dosage frequency adequate to maintain high CD19 occupancy, which may accomplish treatment or prevention of a CD19-associated disorder. In a preferred embodiment, an effective regimen maintains near complete, e.g., greater than 90%, CD19 occupancy on CD19-expressing cells during dosing intervals.

The term “pharmaceutically acceptable” as used herein refers to those compounds, materials, compositions, and/or dosage forms that are, within the scope of sound medical judgment, suitable for contact with the tissues of human beings and animals without excessive toxicity, irritation, allergic response, or other problems or complications commensurate with a reasonable benefit/risk ratio. The term “pharmaceutically compatible ingredient” refers to a pharmaceutically acceptable diluent, adjuvant, excipient, or vehicle with which a CD19-ADC, e.g., SGN-CD19A is administered.

The term “pharmaceutically compatible ingredient” refers to a pharmaceutically acceptable diluent, adjuvant, excipient, or vehicle with which a CD19-ADC, e.g., SGN-CD19A, is administered.

As used herein, the term “about” denotes an approximate range of plus or minus 10% from a specified value. For instance, the language “about 20%” encompasses a range of 18-22%. As used herein, about also includes the exact amount. Hence “about 20%” means “about 20%” and also “20%.

B cell malignancies, also referred to as B-cell lineage malignancies, are treatable by the methods of the present invention. The term B cell malignancies include any malignancy that is derived from a cell of the B cell lineage, including DLBCL and FL. Current treatment for DLBCL and FL includes RCHOP, which is a combination of one monoclonal antibody (rituximab), three chemotherapy agents (cyclophosphamide, doxorubicin, vincristine), and one steroid (prednisone).

The present invention provides, inter alia, methods for treating DLBCL and FL. The present inventors have discovered that combination therapy with an antibody-drug conjugate compounds and chemotherapeutic agents such as RCHP, can improve a therapeutic benefit for subjects suffering from DLBCL and FL. In particular, the present inventors have found that a combination therapy with an anti-CD19 antibody conjugated to an auristatin compound and RCHP provides synergistic therapeutic effects in the treatment of DLBCL and FL. Similarly, the present inventors have found that combination therapy with an anti-CD19 antibody conjugated to an auristatin compound and RCHP provides synergistic therapeutic effects in the treatment of DLBCL and FL. Before the advent of the present invention, it could not have been expected that a combination therapy with an anti-CD19 antibody conjugated to an auristatin compound and RCHP would provide a comparable or more effective treatment of DLBCL and FL in a subject, compared to the current therapy with RCHOP. It also could not have been expected that a combination therapy with an anti-CD19 antibody conjugated to an auristatin compound and RCHP would provide a comparable or more effective treatment of DLBCL and FL in a subject, compared to a combination therapy with an anti-CD19 antibody conjugated to an auristatin compound and RCHOP.

For clarity of disclosure, and not by way of limitation, the detailed description of the invention is divided into the subsections which follow.

### CD19-ADC

A CD19-antibody drug conjugate (CD19-ADC) includes an antibody specific for the human CD19 protein conjugated to a cytotoxic agent. SGN-CD19A is a CD19 ADC produced by the conjugation of the drug-linker intermediate maleimidocaproyl monomethyl auristatin F (mcMMAF) to the humanized antibody hBU12. The points of attachment are cysteines produced by reduction of inter-chain disulfides. SGN-CD19A has an average of four drugs per antibody molecule as shown below:

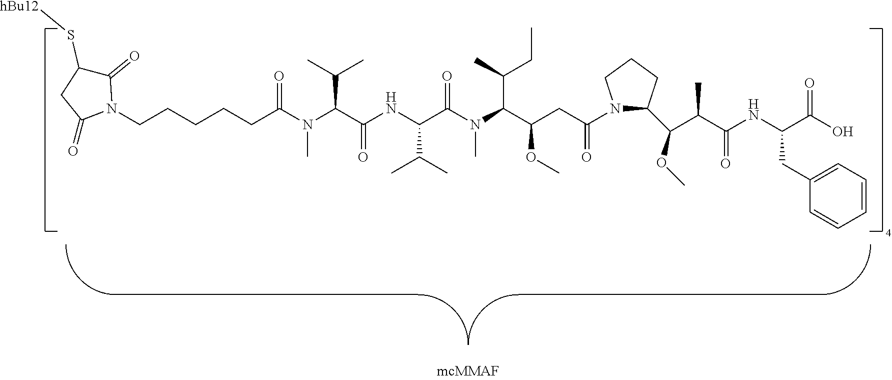

Methods of making the hBU12 antibody are disclosed, e.g., at U.S. Pat. No. 7,968,687. The amino acid sequence of the light chain variable region of hBU12 is provided herein as SEQ ID NO:1. The amino acid sequence of the heavy chain variable region of hBU12 is provided herein as SEQ ID NO:2. hBU12 is an IgG1 antibody and the variable regions are joined to human heavy and light constant regions. U.S. Pat. No. 7,968,687 also provides methods for the synthesis of mcMMAF and its conjugation to hBU12.

SGN-CD19A, therefore, is an ADC that delivers mcMMAF to CD19-positive cells. mcMMAF is a tubulin-binding molecule. SGN-CD19A has a proposed multi-step mechanism of action initiated by binding to its target on the cell surface and subsequent internalization. After cell surface binding, internalization, and trafficking of SGN-CD19A through the endocytic pathway, proteolytic degradation of hBU12 in the lysosomes releases the cysteine adduct of the drug linker in the form of cys-mcMMAF, which then becomes available for tubulin binding. See, e.g., Doronina et al., Nat Biotechnol 21: 778-84 (2003) and Doronina et al., Bioconjug Chem 17: 114-24 (2006). cys-mcMMAF and mcMMAF are used interchangeably herein. Binding of the released drug to tubulin disrupts the cellular microtubule network, leading to G2/M phase cell cycle arrest and subsequent onset of apoptosis in the targeted cell.

### Combination of Chemotherapy and SGN-CD19A to Treat DLBCL and FL

The subject disclosure demonstrates that the combination of either RCHOP with SGN-CD19A or RCHP with SGN-CD19A can be given to subjects at levels that inhibit cancer cell growth, while at the same time are tolerated by the subject. Further, the combination of either RCHOP with SGN-CD19A or RCHP with SGN-CD19A can be effectively administered to achieve antitumor therapeutic effects as a combination at lower levels than either when administered alone. Thus, the combination of either SGN-CD19A with RCHOP or SGN-CD19 with RCHP is synergistic.

In combination with RCHOP or RCHP, SGN-CD19A is administered at a lower level than when used as a single agent. For example in combination with RCHP, SGN-CD19A is administered at a dose between 0.1 and 6.0 mg/kg. Other appropriate dose ranges of SGN-CD19A in combination with RCHOP or RCHP are 0.2 to 4.0 mg/kg, 0.5 to 3.0 mg/kg, and 0.5 to 2.0 mg/kg. Specific appropriate doses of SGN-CD19A in combination with RCHOP or RCHP are 0.5 mg/kg, 1.5 mg/kg, 2.0 mg/kg, 2.5 mg/kg, 3.0 mg/kg, 3.5 mg/kg, 4.0 mg/kg, 4.5 mg/kg, and 5.0 mg/kg, preferably 3.0 mg/kg. In combination with SGN-CD19A, RCHOP or RCHP can also be administered at levels that are less than typical, e.g., one half or one quarter, or one tenth of the usual dose.

This disclosure demonstrates that the combination of RCHOP with SGN-CD19A decreased tumor cell growth to levels similar to or better than those of current RCHOP therapy.

This disclosure also demonstrates that the combination of RCHP with SGN-CD19A decreased tumor cell growth to levels similar to or better than those of SGN-CD19A plus RCHOP combination, or those of current RCHOP therapy. Thus, the combination of SGN-CD19A and RCHP can be used and potentially, result in fewer side effects for patients.

### Administration

SGN-CD19A and a RCHOP or RCHP regimen are administered in such a way that they provide a synergistic effect in the treatment of DLBCL and FL in a patient. Administration can be by any suitable means provided that the administration provides the desired therapeutic effect.

The present invention encompasses treatment schedules wherein the total dosage of SGN-CD19A, administered to a patient with DLBCL and FL will be, for example, 0.1 mg/kg to 6 mg/kg, 0.1 mg/kg to 4 mg/kg, 0.1 mg/kg to 3.2 mg/kg, or 0.1 mg/kg to 2.7 mg/kg of the subject's body weight over a treatment cycle, e.g., a 3 or 4 week time period. In some embodiments, the total dosage of the antibody-drug conjugate compound administered to a patient with DLBCL and FL will be, for example about 0.6 mg/kg to about 6 mg/kg, about 0.6 mg/kg to about 4 mg/kg, about 0.6 mg/kg to about 3.2 mg/kg, about 0.6 mg/kg to about 2.7 mg/kg, or even about 1.5 mg/kg to about 3 mg/kg over a treatment cycle, e.g., a 3 or 6 week time period. In some embodiments, the dosage will be about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, or about 3.8 mg/kg of the subject's body weight over the treatment cycle, e.g., a 3 or 6 week time period. The present invention contemplates administration of the drug for one or more treatment cycles, for example, 1, 2, 3, 4, 5, 6, or more, treatment cycles. In some embodiments, there will be periods of rest between one or more of the treatment cycles. For example, in some embodiments, there will be a period of rest between the second and third treatment cycle but not the first and second treatment cycle. In another embodiment, there might be a period of rest between the first and second treatment cycle but not the second and third treatment cycle. Dosing schedules include, for example, administering SGN-CD19A once during a treatment cycle, e.g., on day 1 of a 21-day cycle. Other dosage schedules are encompassed by the present invention. In preferred embodiments, SGN-CD19A is administered once on day 1 of the first, third and fifth 21-day cycle, and skipped in the second, fourth and sixth 21-day cycle, for a maximum of 3 doses.

It will be readily apparent to those skilled in the art that other SGN-CD19A doses or frequencies of administration that provide the desired synergistic effect in combination with RCHP are suitable for use in the present invention.

Administration of SGN-CD19A and RCHOP or RCHP can be on the same or different days provided that administration provides the desired therapeutic effect.

In some embodiments, RCHOP or RCHP will be administered at levels currently indicated in the art for the treatment of DLBCL and FL or at lower or higher levels than those currently indicated in the art for the treatment of DLBCL and FL provided that such dosage provides the desired therapeutic effect. Embodiments of the present invention include, for example, those wherein RCHOP or RCHP is administered at about the MTD, maximum tolerated dose. The present invention contemplates administration of RCHOP or RCHP for one or more treatment cycles, for example, 1, 2, 3, 4, 5, 6, or more treatment cycles. It will be understood that any of the dose ranges indicated herein for treatment with RCHOP or RCHP can be combined with any of the dose ranges indicated herein for treatment DLBCL and FL provided that administration provides the desired therapeutic effect.

In some particularly preferred examples of the present invention, administration of a synergistic amount of SGN-CD19A includes once during the treatment cycle (e.g., a 21-day treatment cycle) in a range of about 0.5 to about 6.0 mg/kg, about 0.6 mg/kg to about 4.0 mg/kg, about 0.8 mg/kg to about 4.0 mg/kg, about 1.8 mg/kg to about 4.0 mg/kg, about 1 mg/kg to about 3.5 mg/kg, about 1.5 mg/kg to about 3.5 mg/kg, or even more preferably about 2.5 mg/kg to about 3.5 mg/kg, or at a dose of about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, or about 5.0 mg/kg, more preferably about 3.0 mg/kg of the subject's body weight in combination with administering RCHOP or RCHP at standard dosing schedules known in the art. In preferred embodiments, SGN-CD19A is administered once on day 1 of the first, third and fifth 21-day cycle, and skipped in the second, fourth and sixth 21-day cycle, for a maximum of 3 doses.

Administration of a synergistic amount of the therapeutic agents RCHP includes administering rituximab once during the treatment cycle (e.g., a 21 day treatment cycle), in a range of about 100 mg/m² to about 500 mg/m², about 100 mg/m² to about 450 mg/m², about 100 mg/m² to about 400 mg/m², about 100 mg/m² to about 350 mg/m², about 100 mg/m² to about 300 mg/m², about 200 mg/m² to about 400 mg/m², about 250 mg/m² to about 400 mg/m², about 300 mg/m² to about 400 mg/m², or about 350 mg/m² to about 400 mg/m², or at a dose of about 100 mg/m², about 150 mg/m², about 200 mg/m², about 250 mg/m², about 300 mg/m², about 350 mg/m², about 400 mg/m², or about 450 mg/m², more preferably about 375 mg/m² of the body surface area of the subject; administering cyclophosphamide once during the treatment cycle (e.g., a 21 day treatment cycle), in a range of about 400 mg/m² to about 800 mg/m², about 400 mg/m² to about 750 mg/m², about 400 mg/m² to about 600 mg/m², about 400 mg/m² to about 550 mg/m², about 400 mg/m² to about 500 mg/m², about 500 mg/m² to about 800 mg/m², about 550 mg/m² to about 800 mg/m², about 600 mg/m² to about 800 mg/m², or about 650 mg/m² to about 800 mg/m², or at a dose of about 400 mg/m², about 450 mg/m², about 500 mg/m², about 550 mg/m², about 600 mg/m², about 650 mg/m², about 700 mg/m², or about 750 mg/m², more preferably about 750 mg/m² of the body surface area of the subject; administering doxorubicin once during the treatment cycle (e.g., a 21 day treatment cycle), in a range of about 20 mg/m² to about 60 mg/m², about 20 mg/m² to about 55 mg/m², about 20 mg/m² to about 50 mg/m², about 20 mg/m² to about 45 mg/m², about 20 mg/m² to about 40 mg/m², about 30 mg/m² to about 60 mg/m², about 35 mg/m² to about 60 mg/m², about 40 mg/m² to about 60 mg/m², or about 45 mg/m² to about 55 mg/m², or at a dose of about 10 mg/m², about 15 mg/m², about 20 mg/m², about 25 mg/m², about 30 mg/m², about 35 mg/m², about 40 mg/m², about 45 mg/m², about 50 mg/m², or about 55 mg/m², more preferably about 50 mg/m² of the body surface area of the subject; administering prednisone once a day on days 1 to 5 during the treatment cycle (e.g., a 21 day treatment cycle), in a range of about 50 mg to about 100 mg, about 60 mg to about 100 mg, about 70 mg to about 100 mg, about 80 mg to about 100 mg, or about 90 mg to about 100 mg, or at a dose of about 50 mg, about 60 mg, about 70 mg, about 80 mg, about 90 mg, about 95 mg, or about 100 mg, more preferably about 100 mg, in combination with administering SGN-19A at dosing schedules disclosed herein. In preferred embodiments, amount of the therapeutic agents RCHP is administered as follows: 375 mg/m² of rituximab once on day 1 of a 21 day treatment cycle; 750 mg/m² of cyclophosphamide once on day 1 of a 21 day treatment cycle; 50 mg/m² of doxorubicin once on day 1 of a 21 day treatment cycle; and 100 mg of prednisone once a day on days 1 to 5 of a 21 day treatment cycle, for a maximum of 6 treatment cycles.

Administration of a synergistic amount of the therapeutic agents RCHOP includes administering rituximab once during the treatment cycle (e.g., a 21 day treatment cycle), in a range of about 100 mg/m² to about 500 mg/m², about 100 mg/m² to about 450 mg/m², about 100 mg/m² to about 400 mg/m², about 100 mg/m² to about 350 mg/m², about 100 mg/m² to about 300 mg/m², about 200 mg/m² to about 400 mg/m², about 250 mg/m² to about 400 mg/m², about 300 mg/m² to about 400 mg/m², or about 350 mg/m² to about 400 mg/m², or at a dose of about 100 mg/m², about 150 mg/m², about 200 mg/m², about 250 mg/m², about 300 mg/m², about 350 mg/m², about 400 mg/m², or about 450 mg/m², more preferably about 375 mg/m² of the body surface area of the subject; administering cyclophosphamide once during the treatment cycle (e.g., a 21 day treatment cycle), in a range of about 400 mg/m² to about 800 mg/m², about 400 mg/m² to about 750 mg/m², about 400 mg/m² to about 600 mg/m², about 400 mg/m² to about 550 mg/m², about 400 mg/m² to about 500 mg/m², about 500 mg/m² to about 800 mg/m², about 550 mg/m² to about 800 mg/m², about 600 mg/m² to about 800 mg/m², or about 650 mg/m² to about 800 mg/m², or at a dose of about 400 mg/m², about 450 mg/m², about 500 mg/m², about 550 mg/m², about 600 mg/m², about 650 mg/m², about 700 mg/m², or about 750 mg/m², more preferably about 750 mg/m² of the body surface area of the subject; administering doxorubicin once during the treatment cycle (e.g., a 21 day treatment cycle), in a range of about 20 mg/m² to about 60 mg/m², about 20 mg/m² to about 55 mg/m², about 20 mg/m² to about 50 mg/m², about 20 mg/m² to about 45 mg/m², about 20 mg/m² to about 40 mg/m², about 30 mg/m² to about 60 mg/m², about 35 mg/m² to about 60 mg/m², about 40 mg/m² to about 60 mg/m², or about 45 mg/m² to about 55 mg/m², or at a dose of about 10 mg/m², about 15 mg/m², about 20 mg/m², about 25 mg/m², about 30 mg/m², about 35 mg/m², about 40 mg/m², about 45 mg/m², about 50 mg/m², or about 55 mg/m², more preferably about 50 mg/m² of the body surface area of the subject; administering vincristine once during the treatment cycle (e.g., a 21 day treatment cycle), in a range of about 0.5 mg/m² to about 3.0 mg/m², about 0.5 mg/m² to about 2.5 mg/m², about 0.5 mg/m² to about 2.0 mg/m², about 0.5 mg/m² to about 1.5 mg/m², about 1.0 mg/m² to about 3.0 mg/m², about 1.0 mg/m² to about 2.0 mg/m², preferably about 1.0 mg/m² to about 1.5 mg/m², more preferably about 1.4 mg/m² of the body surface area of the subject; administering prednisone once a day on days 1 to 5 during the treatment cycle (e.g., a 21 day treatment cycle), in a range of about 50 mg to about 500 mg, about 50 mg to about 400 mg, about 50 mg to about 300 mg, about 50 mg to about 200 mg, 50 mg to about 150 mg, about 75 mg to about 125 mg, about 90 mg to about 110 mg, about 50 mg to about 100 mg, about 60 mg to about 100 mg, about 70 mg to about 100 mg, about 80 mg to about 100 mg, or about 90 mg to about 100 mg, or at a dose of about 50 mg, about 60 mg, about 70 mg, about 80 mg, about 90 mg, about 95 mg, or about 100 mg, more preferably about 100 mg, in combination with administering SGN-19A at dosing schedules disclosed herein. In preferred embodiments, amount of the therapeutic agents RCHOP is administered as follows: 375 mg/m² of rituximab once on day 1 of a 21 day treatment cycle; 750 mg/m² of cyclophosphamide once on day 1 of a 21 day treatment cycle; 50 mg/m² of doxorubicin once on day 1 of a 21 day treatment cycle; 1.4 mg/m² of vincristine once on day 1 of a 21 day treatment cycle; and 100 mg of prednisone once a day on days 1 to 5 of a 21 day treatment cycle, for a maximum of 6 treatment cycles.

### Subjects

The methods of the present invention encompass administering combination therapy to a subject for the treatment of DLBCL and FL.

The subjects to be treated with the methods of the present invention are those that have been diagnosed with DLBCL or FL or are suspected of having DLBCL or FL. Diagnosis can be by methods known in the art, including, identification of immature white blood cells (lymphoblasts) in peripheral blood or bone marrow.

The methods of the present invention encompass treating a subject who is newly diagnosed and has not previously been treated for DLBCL or FL.

The methods of the present invention also can be used to treat subjects with refractory and/or relapsed DLBCL or FL. A subject with refractory DLBCL or FL is a subject who does not respond to therapy for DLBCL or FL, i.e., the subject continues to experience disease progresssion despite therapy. A subject with relapsed DLBCL or FL is a subject who has responded to therapy for DLBCL or FL at one point, but has had a reoccurrence or further progression of disease following the response.

The methods of the present invention also encompass treating a subject who has previously undergone a stem cell transplant.

### EXAMPLES

The following examples are offered to illustrate, but not to limit the claimed invention.

### Example 1. Denintuzumab Mafodotin (SGN-CD19A) Combined With RCHOP or RCHP versus RCHOP Alone in Diffuse Large B-Cell Lymphoma or Follicular Lymphoma

This is a Phase 2 study to evaluate the combination of denintuzumab mafodotin in combination with RCHOP or RCHP compared with RCHOP alone as front-line therapy in patients with diffuse large B-cell lymphoma or follicular lymphoma Grade 3b.

Detailed Description: In Part A of the study, patients will be randomized 1:1 to receive denintuzumab mafodotin plus RCHOP (rituximab, cyclophosphamide, doxorubicin, vincristine, and prednisone) or denintuzumab mafodotin plus RCHP (rituximab, cyclophosphamide, doxorubicin, and prednisone) to assess the safety of these 2 combination regimens. Part B of the study is designed to evaluate the antitumor activity and safety of denintuzumab mafodotin in combination with either RCHOP or RCHP (Experimental Arm) compared with RCHOP alone (Comparator Arm).

### Inclusion Criteria

Treatment-naive patients with histologically confirmed systemic de novo or transformed diffuse large B-cell lymphoma (DLBCL) (from follicular or marginal zone lymphoma), or follicular lymphoma (FL) Grade 3b;
patients must have high intermediate or high risk disease based on standard International Prognostic Index (IPI) (score ≥3 for patients >60 years of age) or age-adjusted IPI (aaIPI) (score 2 or 3 for patients ≤60 years of age), and stage IAX (bulk defined as single lymph node mass >10 cm in diameter)—IV disease
Tumor tissue available from most recent biopsy to determine cell of origin.
Fluorodeoxyglucose-avid disease by positron emission tomography and measurable disease greater than 1.5 cm diameter.
Eastern Cooperative Oncology Group performance status ≤2.
Age 18 years or older.
Adequate study baseline laboratory parameters.

### Exclusion Criteria

Previous history of treated indolent lymphoma.
History of another primary invasive cancer, hematologic malignancy, or myelodysplastic syndrome that has not been in remission for at least 3 years.
History of progressive multifocal leukoencephalopathy.
Cerebral/meningeal disease related to the underlying malignancy.
Patients with the following ocular conditions: corneal disorders, monocular vision (ie. best corrected visual acuity greater than or equal to 20/200 in one eye), or active ocular disorders requiring treatment.

### Part A

denintuzumab mafodotin (SGN-CD19A)+RCHOP (rituximab, cyclophosphamide, doxorubicin, vincristine, and prednisone)

denintuzumab mafodotin—3 mg/kg will be administered every 6 weeks via intravenous (IV) infusion, up to a maximum of three (3) doses, on Day 1 of Cycles 1, 3, and 5 of 21-day cycles
rituximab—375 mg/m² every 3 weeks by IV infusion for up to 6 cycles
cyclophosphamide—750 mg/m² every 3 weeks by IV infusion for up to 6 cycles
doxorubicin—50 mg/m² every 3 weeks by IV infusion for up to 6 cycles
vincristine—1.4 mg/m² every 3 weeks by IV infusion for up to 6 cycles (dose capped at 2 mg total)
prednisone—100 mg on Days 1 to 5 of each 3-week cycle, orally for up to 6 cycles

denintuzumab mafodotin (SGN-CD19A)+RCHP (rituximab, cyclophosphamide, doxorubicin, and prednisone)

denintuzumab mafodotin—3 mg/kg will be administered every 6 weeks via intravenous (IV) infusion, up to a maximum of three (3) doses, on Day 1 of Cycles 1, 3, and 5 of 21-day cycles
rituximab—375 mg/m² every 3 weeks by IV infusion for up to 6 cycles
cyclophosphamide—750 mg/m² every 3 weeks by IV infusion for up to 6 cycles
doxorubicin—50 mg/m² every 3 weeks by IV infusion for up to 6 cycles
prednisone—100 mg on Days 1 to 5 of each 3-week cycle, orally for up to 6 cycles

### Part B

denintuzumab mafodotin (SGN-CD19A)+RCHOP denintuzumab mafodotin—3 mg/kg will be administered every 6 weeks via intravenous (IV) infusion, up to a maximum of three (3) doses, on Day 1 of Cycles 1, 3, and 5 of 21-day cycles

rituximab—375 mg/m² every 3 weeks by IV infusion for up to 6 cycles
cyclophosphamide—750 mg/m² every 3 weeks by IV infusion for up to 6 cycles
doxorubicin—50 mg/m² every 3 weeks by IV infusion for up to 6 cycles
vincristine—1.4 mg/m² every 3 weeks by IV infusion for up to 6 cycles (dose capped at 2 mg total)
prednisone—100 mg on Days 1 to 5 of each 3-week cycle, orally for up to 6 cycles

denintuzumab mafodotin (SGN-CD19A)+RCHP

denintuzumab mafodotin—3 mg/kg will be administered every 6 weeks via intravenous (IV) infusion, up to a maximum of three (3) doses, on Day 1 of Cycles 1, 3, and 5 of 21-day cycles
rituximab—375 mg/m² every 3 weeks by IV infusion for up to 6 cycles
cyclophosphamide—750 mg/m² every 3 weeks by IV infusion for up to 6 cycles
doxorubicin—50 mg/m² every 3 weeks by IV infusion for up to 6 cycles
prednisone—100 mg on Days 1 to 5 of each 3-week cycle, orally for up to 6 cycles

RCHOP alone: (rituximab, cyclophosphamide, doxorubicin, vincristine, and prednisone)

rituximab—375 mg/m² every 3 weeks by IV infusion for up to 6 cycles
cyclophosphamide—750 mg/m² every 3 weeks by IV infusion for up to 6 cycles
doxorubicin—50 mg/m² every 3 weeks by IV infusion for up to 6 cycles
vincristine—1.4 mg/m² every 3 weeks by IV infusion for up to 6 cycles (dose capped at 2 mg total)
prednisone—100 mg on Days 1 to 5 of each 3-week cycle, orally for up to 6 cycles

### Outcome Measures

### 1. Complete Response Rate (CRR) in Part B

2. Incidence of adverse events and laboratory abnormalities in Part A and Part B.
3. Event-free survival (EFS) between study arms in Part B.
4. Progression-free survival (PFS) between study arms in Part B.
5. Overall survival (OS) between study arms in Part B.
6. Objective response rate (ORR) at End Of Treatment (EOT) between study arms in Part B.
7. Duration of objective response (OR) and of complete response (CR) between study arms in Part B.

### Results

Progression-free survival (PFR) is determined (until disease progression, subsequent anticancer chemotherapy, death, or study closure, up to 5 years posttreatment). Complete response rate (CRR), Event-free survival (EFS), overall survival (OS), and objective response rate (ORR), as well as duration of objective response (OR) and of complete response (CR) between study arms are also evaluated.

It is surprisingly found that patients treated with the combination of denintuzumab mafodotin (SGN-CD19A)+RCHP (rituximab, cyclophosphamide, doxorubicin, and prednisone) exhibit one or more of improved CRR, PFR, EFS, OS or ORR, or duration of OR and of CR, compared to patients treated with RCHOP (rituximab, cyclophosphamide, doxorubicin, vincristine, and prednisone). It is also surprisingly found that patients treated with the combination of denintuzumab mafodotin (SGN-CD19A)+RCHP (rituximab, cyclophosphamide, doxorubicin, and prednisone) exhibit one or more of improved CRR, PFR, EFS, OS or ORR, or duration of OR and of CR, compared to patients treated with the combination of denintuzumab mafodotin (SGN-CD19A)+RCHOP (rituximab, cyclophosphamide, doxorubicin, vincristine, and prednisone).

It is further surprisingly found that patients treated with the combination of denintuzumab mafodotin (SGN-CD19A)+RCHOP (rituximab, cyclophosphamide, doxorubicin, vincristine and prednisone) exhibit one or more of improved CRR, PFR, EFS, OS or ORR, or duration of OR and of CR, compared to patients treated with RCHOP (rituximab, cyclophosphamide, doxorubicin, vincristine, and prednisone).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating a subject having Diffuse Large B-cell Lymphoma (DLBCL) or Follicular Lymphoma (FL), the method comprising administering to the subject a drug combination consisting essentially of a CD19 antibody drug conjugate (CD19-ADC) , and rituximab, cyclophosphamide, doxorubicin, and prednisone (RCHP).

2. The method of claim 1, wherein the CD19-ADC comprises a humanized hBU12 antibody conjugated to a maleimidocaproyl monomethyl auristatin F (mcMMAF) molecule.

3. The method of claim 1, wherein the CD19-ADC is SGN-CD19A.

4. The method of claim 1, wherein Diffuse Large B-cell Lymphoma (DLBCL) or Follicular Lymphoma (FL) in the subject has not been previously treated.

5. The method of claim 1, wherein the CD19-ADC is administered at a dosage between 0.5 and 6.0 mg/kg based on the weight of the subject.

6. The method of claim 1, wherein the CD19-ADC is administered at a dosage of about 3.0 mg/kg based on the weight of the subject.

7. The method of claim 1, wherein the CD19-ADC is administered once every 6 weeks.

8. The method of claim 1, wherein the CD19-ADC is administered on day 1 of the 6-week cycle.

9. The method of claim 1, wherein rituximab is administered at a dosage between about 350 mg/m² to about 400 mg/m² based on the body surface area of the subject.

10. The method of claim 9, wherein rituximab is administered at a dosage of about 375 mg/m² based on the body surface area of the subject.

11. The method of claim 9, wherein rituximab is administered once every 3 weeks.

12. (canceled)

13. The method of claim 1, wherein cyclophosphamide is administered at a dosage between about 700 mg/m² to about 800 mg/m² based on the body surface area of the subject.

14. The method of claim 13, wherein cyclophosphamide is administered at a dosage of about 750 mg/m² based on the body surface area of the subject.

15. The method of claim 13, wherein cyclophosphamide is administered once every 3 weeks.

16. (canceled)

17. The method of claim 1, wherein doxorubicin is administered at a dosage between about 40 mg/m² to about 60 mg/m² based on the body surface area of the subject.

18. The method of claim 17, wherein doxorubicin is administered at a dosage of about 50 mg/m² based on the body surface area of the subject.

19. The method of claim 17, wherein doxorubicin is administered once every 3 weeks.

20. (canceled)

21. The method of claim 1, wherein prednisone is administered at a dosage between about 80 mg to about 100 mg.

22. The method of claim 21, wherein prednisone is administered at a dosage of about 100 mg.

23. The method of claim 20, wherein prednisone is administered multiple times during a 3-week cycle.

24. (canceled)
